(19) United States
(12) Patent Application Publication
(10) Pub. No.: US 2021/0050098 A1
(43) Pub. Date: Feb. 18, 2021

(54) Post-Operative Monitoring Via Patient Reported Outcomes

(71) Applicant: Avent, Inc., Alpharetta, GA (US)

(72) Inventors: Jeanette Sterner, Eagan, MN (US); Arne Jørgen Madsen, Newport Beach, CA (US); Kathy M. Muir, Rochester Hills, MI (US); Julyana Z. Tanudjaja, Cumming, GA (US)

(21) Appl. No.: 16/969,402

(22) PCT Filed: Feb. 13, 2019
(86) PCT No.: PCT/US2019/017851

§ 371 Date: Aug. 12, 2020

Related U.S. Application Data
(60) Provisional application No. 62/710,464, filed on Feb. 16, 2018.

(51) Int. Cl. G16H 40/20; G16H 10/60; G16H 50/70; G16H 15/00; G16H 10/20; A61B 5/00

(52) U.S. Cl. CPC G16H 40/20; G16H 10/60; A61B 5/7475; G16H 15/00; G16H 10/20; G16H 50/70

(57) ABSTRACT

Methods and systems for managing patient health data are provided. A patient data tracking system is provided that aggregates patient data to create one or more patient data sets. The system analyzes and/or sorts the patient data set(s) to selectively provide information to different entities. As one example, the system can generate a benchmark for one or more patient outcomes from the aggregate patient data, and the aggregate benchmark(s) can be compared to patient outcomes for a patient population, i.e., a subset of the aggregate patient data, to help a physician and/or healthcare organization monitor patient outcomes, develop treatment protocols for a patient population, or the like. Further, the system may generate one or more notifications to a caregiver based on patient outcomes, which can enable proactive intervention and follow-up with the patient and thereby help improve patient care and outcomes.

### CROSS-REFERENCE TO RELATED APPLICATIONS

The present application claims priority to U.S. Provisional Application Ser. No. 62/710,464, filed on Feb. 16, 2018, which is incorporated herein in its entirety by reference thereto.

### FIELD

The subject matter of the present disclosure relates generally to methods and systems for managing patient health data. More particularly, the present subject matter is directed to methods and systems for monitoring and utilizing patient health data to improve patient outcomes.

### BACKGROUND

Healthcare providers and organizations, as well as patients, continuously strive to improve patient outcomes and quality of care. Moreover, healthcare is increasingly focusing on value-based care, with penalties for poor patient satisfaction, hospital readmissions, and emergency room (ER) visits after surgery. However, historically, it has been difficult for clinicians (e.g., surgeons, anesthesiologists) and hospital administrators to track patients' post-operative recovery progress after discharge. In the value-based care environment, it is imperative that clinicians get real-time or near real-time feedback from their patients such that clinicians can better direct patient outcomes. For example, by receiving real-time or near real-time feedback related to therapies and/or devices, clinicians could better direct patient outcomes related to, e.g., post-surgical pain management, enteral feeding, respiratory airway management, and surgical infection prevention. Further, knowing how their patients compare to benchmarks derived from an aggregate patient data set could help clinicians and healthcare organizations better monitor and direct patient outcomes.

Consequently, there is a need for one or more methods and systems for monitoring patient outcomes. In particular, a method and system for generating user notifications upon receipt of patient health data would be beneficial. Additionally, a method and system for benchmarking patient health data and comparing patient health data to an aggregate data set would be useful.

### SUMMARY

The present invention provides methods and systems for managing patient health data. In particular, the present invention provides a patient health platform that collects patient-generated data, e.g., from patients, physicians, device sensors, and the like, to create one or more patient data sets. The platform analyzes and/or sorts the patient data set(s) such that the platform may selectively provide targeted or specific information to physicians, patients, healthcare organizations, medical and other device manufacturers, and/or payors. Sorting the patient data set(s) may include securing the patient data such that the platform includes features for storing and disseminating the patient data set(s) in compliance with one or more applicable governmental or organizational regulations. As one example, the platform can provide targeted information to a physician and healthcare organization that can help the physician and healthcare organization direct a patient's pain management therapy, develop treatment protocols for a patient population, or the like. As a further example, the platform can generate a benchmark for one or more patient outcomes from the aggregate patient data, and the aggregate benchmark(s) can be compared to patient outcomes for a patient population, i.e., a subset of the aggregate patient data, to help a physician and/or healthcare organization monitor patient outcomes, develop treatment protocols for a patient population, etc. The platform may use one or more dashboards to disseminate targeted information to physicians, patients, healthcare organizations, medical or other device manufacturers, payors, and/or other appropriate recipients. The dashboards may present information regarding an individual patient or one or more patient populations, e.g., aggregate data or trends regarding a patient population.

Moreover, the platform may generate one or more notifications to a caregiver based on patient outcomes, which can enable proactive intervention and follow-up with the patient and thereby help improve patient care and outcomes. Additional aspects and advantages of the invention will be set forth in part in the following description, may be apparent from the description, or may be learned through practice of the invention.

In one aspect, the present subject matter is directed to a system for monitoring patient outcomes. The system comprises a data device for inputting patient data. The system further comprises a data administration tool for aggregating the patient data of all patients inputting patient data into the system to form an aggregate patient data set and for generating an aggregate benchmark for a patient outcome from the aggregate patient data set. The system also comprises a dashboard for disseminating a comparison of the aggregate benchmark to a summary of the data for the patient outcome of a patient population. The data for the patient outcome of the patient population is a subset of the aggregate patient data. In some embodiments, the data administration tool comprises an analysis module that generates the aggregate benchmark; the analysis module may also generate the summary. Moreover, the patient outcome may be patient data reported by the patient.

Further, certain embodiments of the system comprise a notification engine for generating one or more notifications to a caregiver. The notification engine is configured to generate a notification when the patient outcome exceeds a threshold. The threshold may be selected by the caregiver. In some embodiments, the data device is a patient data device. A patient may input patient data into a web-based survey accessed through the patient data device. In other embodiments, the system comprises a plurality of data devices and a plurality of dashboards.

In another aspect, the present subject matter is directed to a method for tracking patient outcomes. The method comprises receiving patient data through patient inputs to a data collection protocol; aggregating the patient data to form an aggregated patient data set; generating a first aggregate benchmark for a first patient outcome of the aggregated patient data set; and disseminating a comparison of a summary of the first patient outcome for a patient population and the first aggregate benchmark. In some embodiments, disseminating the comparison comprises disseminating the comparison to a dashboard. Moreover, the data collection protocol completed by a patient may comprise at least one survey question selected by the patient's caregiver.

The method also may comprise generating a second aggregate benchmark for a second patient outcome of the aggregated patient data set and disseminating a comparison of a summary of the second patient outcome for the patient population and the second aggregate benchmark. In particular embodiments, disseminating the comparison of the summary of the second patient outcome and the second aggregate benchmark comprises disseminating the comparison of the summary of the second patient outcome and the second aggregate benchmark to the dashboard. Further, the dashboard may display the comparison of the summary of the first patient outcome and the first aggregate benchmark alongside the comparison of the summary of the second patient outcome and the second aggregate benchmark.

In some embodiments, the method also comprises generating a notification based on the patient data. In one embodiment, the notification is an individual notification that is generated based on a single patient outcome. In another embodiment, the notification is a group notification that is generated based on a group of patient outcomes. The notification, whether it is an individual or group notification, may be generated when one or more patient outcomes meet or exceed thresholds for the one or more patient outcomes. The thresholds may be selected by a caregiver, e.g., a physician receiving the notification.

The method also may comprise securing the patient data against tampering or unauthorized access. In addition, the method may comprise controlling access to the patient data. For example, one entity may access only a portion of the patient data, e.g., completely de-identified patient data, while another entity may access a larger portion of the patient data, e.g., patient data with some personal identifiers. Access to the patient data may be controlled in other ways as well.

In yet another aspect, the present subject matter is directed to a method for administering patient data. The method comprises establishing a survey protocol; providing a patient access to the survey protocol; and collecting patient data inputs through the survey protocol. The patient data inputs represent a plurality of patient outcomes. The method further comprises aggregating patient data inputs collected from a plurality of patients; generating an aggregate benchmark for a patient outcome of the aggregated patient data inputs; and disseminating a comparison of a summary of the patient outcome for a patient population and the aggregate benchmark.

In some embodiments, the method also comprises determining a patient procedure for a patient population; configuring, based on the patient procedure, the survey protocol for collecting patient inputs; presenting the patient population with prompts; and receiving patient data inputs based on the prompts. Configuring the survey protocol may comprise including questions or prompts in the survey protocol selected by a caregiver of the patient population.

In some embodiments, the method also comprises generating a notification based on the patient data. In one embodiment, the notification is an individual notification that is generated based on a single patient outcome. In another embodiment, the notification is a group notification that is generated based on a group of patient outcomes. The notification, whether it is an individual or group notification, may be generated when one or more patient outcomes meet or exceed thresholds for the one or more patient outcomes. The thresholds may be selected by a caregiver, e.g., a physician receiving the notification.

Additionally, in other aspects, the platform comprises a method and/or system, such as a mobile application (i.e., a mobile app), for collecting patient-generated data. In another embodiment, the system comprises a web-based data collection protocol for collecting patient-generated data. Other telecommunications systems, devices, or interfaces also may be used to collect patient-generated data. The platform also may comprise an interface for collecting data derived from sensors connected to patient devices, e.g., an infusion pump or the like, or worn by patients, e.g., a wearable heart rate monitor or the like. As appropriate, the platform may include a method and/or system for integrating the data from the patient devices and/or sensors worn by patients with the patient-generated data. In other embodiments, the platform also may comprise a method and/or system for integrating medical procedure information as well as data from patients' electronic medical records (“EMR”) and/or other systems, e.g., from an existing EMR or PAS system, with the patient-generated, patient device, and/or wearable sensor data.

In still other embodiments, the platform secures the patient data in a manner that complies with applicable governmental or organizational regulations. For example, the platform may comply with the requirements of the Health Insurance

Portability and Accountability Act (“HIPAA”) such that the platform is HIPAA-compliant. It should be understood that the patient health platform, including one or more methods and/or systems for collecting, analyzing, storing, and disseminating patient health data, may be further configured with any of the additional features as described herein.

These and other features, aspects, and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1A provides a diagram view of a portion of a representative tracking system for a patient tracking system platform in accordance with an exemplary embodiment of the present subject matter.

FIG. 1B provides a diagram view of another portion of the representative tracking system of FIG. 1B.

FIG. 2 provides a chart illustrating a method for tracking patient outcomes according to an exemplary embodiment of the present subject matter.

FIG. 3 provides a chart illustrating a method for tracking patient outcomes according to another exemplary embodiment of the present subject matter.

FIG. 4 provides a chart illustrating a method for administering patient data according to an exemplary embodiment of the present subject matter.

FIG. 5 provides a graph illustrating a comparison between an aggregate benchmark and a summary of a patient outcome for a patient population according to an exemplary embodiment of the present subject matter.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

In general, the present disclosure is directed to methods and systems for tracking or monitoring patient outcomes, particularly for the development and/or modification of patient therapies, the management and improvement of medical devices or instruments, and/or the management and improvement of healthcare sites or facilities. Preferably, the methods and systems are computer-implemented, e.g., using one or more Internet-based interfaces. For sake of example only, the following discussion relates to embodiments of the invention drawn to healthcare-related patient outcome tracking platforms. It should be appreciated, however, that the systems and methods are just as applicable to any manner of tracking platforms.

Embodiments of the methods and system disclosed herein may be executed by one or more suitable networked systems, such as, e.g., personal computers, handheld devices, medical devices, databases, and the like. Such system(s) may comprise one or more computing devices adapted to perform one or more embodiments of the methods disclosed herein. Such systems and computing devices may access one or more computer-readable media that embody computer-readable instructions which, when executed by at least one computer, cause the computer(s) to implement one or more embodiments of the methods of the present subject matter. Additionally or alternatively, the computing device(s) may comprise circuitry that renders the device(s) operative to implement one or more of the methods of the present subject matter. Further, components of the presently-disclosed technology may be implemented using one or more computer-readable media. Any suitable computer-readable medium or media may be used to implement or practice the presently-disclosed subject matter, including, but not limited to, diskettes, drives, and other magnetic-based storage media, optical storage media, including disks (including CD-ROMS, DVD-ROMS, and variants thereof), flash, RAM, ROM, and other memory devices, and the like. In addition, to the extent one or more computer-implemented programs are used, the present subject matter is not limited to any particular programming language. It should be understood that a variety of programming languages may be used to implement the present subject matter as described herein, and any references to specific languages are provided by way of illustrative example only.

The present disclosure also makes reference to the transmission of communicated data over one or more communications networks. It should be appreciated that network communications can comprise sending and/or receiving information over one or more networks of various forms. For example, a network can comprise a dial-in network, a local area network (“LAN”), a wide area network (“WAN”), a public switched telephone network (“PSTN”), the Internet, an intranet, or other type(s) of networks. A network may comprise any number and/or combination of hard-wired, wireless, or other communication links.

Moreover, the particular the naming of the components, capitalization of terms, the attributes, data structures, or any other programming or structural aspect is not mandatory or significant, and the mechanisms that implement the invention or its features may have different names, formats, or protocols. Moreover, the systems may comprise and the methods may be implemented via a combination of hardware and software, as described, or entirely in hardware elements. Also, the particular division of functionality between the various components described herein is merely exemplary and not mandatory; functions performed by a single component may instead be performed by multiple components, and functions performed by multiple components may instead performed by a single component.

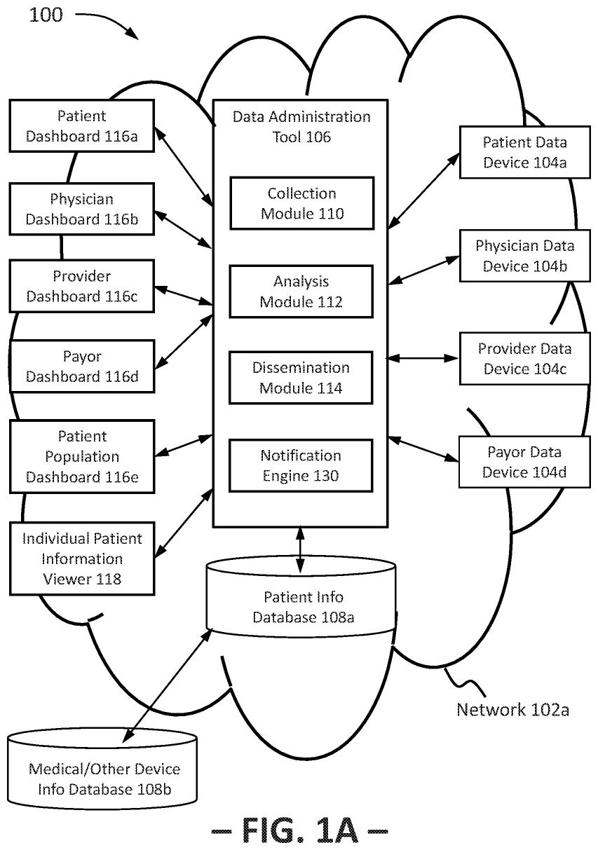

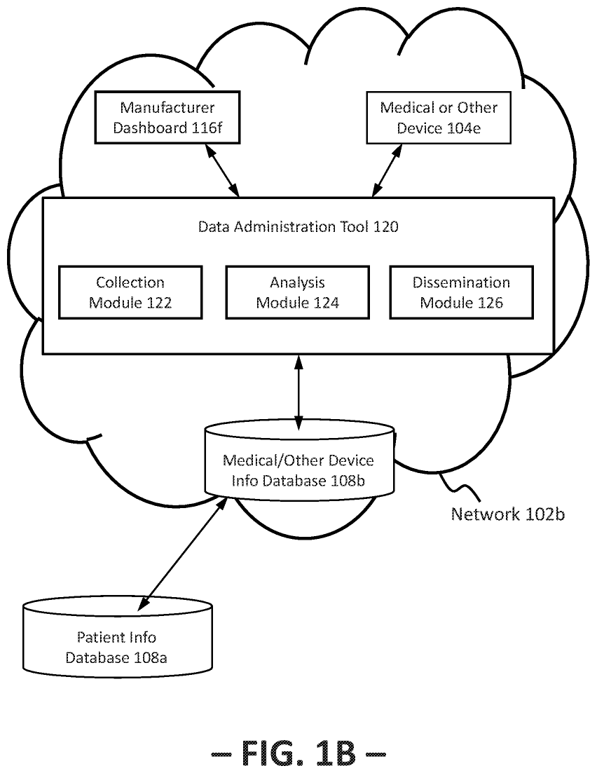

FIG. 1A provides a diagram view of a representative tracking system 100 that may be used to practice aspects of the patient tracking system platform in accordance with an exemplary embodiment of the present subject matter. Tracking system 100 includes a network 102 for sending and/or receiving information or data as previously described. A data device 104 connected through network 102 to a data administration tool 106 may provide patient data to the data administration tool 106 for use in tracking a patient's outcomes. In some embodiments, the tracking system 100 may include a plurality of data devices 104. For example, as shown in FIG. 1A, a patient may use a patient data device 104a to provide patient-generated data. As further shown in FIG. 1A, a physician may provide physician-generated patient data through a physician data device 104b; a healthcare provider, such as a hospital or other healthcare organization, may provide provider-generated patient data through a provider data device 104c; and a payor organization, such as an insurance company, may provide payor-generated patient data through a payor data device 104d. Additionally or alternatively, each of a patient, a physician, a provider, and a payor may receive patient data through the respective data device 104a, 104b, 104c, 104d. Patient data device 104a, physician data device 104b, provider data device 104c, and payor data device 104d may comprise a personal computing device, such as portable or mobile telecommunications devices, e.g., with Internet functionality. As examples, data devices 104a, 104b, 104c, 104d may be desktop computers, tablet computers, smartphones, or any other suitable personal computing devices. Moreover, one or more medical or other devices or instruments 104e also may be connected to data administration tool 106, or a separate data administration tool 120, to provide patient data for use in, e.g., tracking patient outcomes, developing treatment protocols, refining the configuration or functionality of the medical or other device, etc. Tracking system 100 also may comprise other data devices 104.

The data devices 104 are configured to execute one or more computer programs, such as an Internet browser program, to allow users to interact with the data administration tool 106, a data collection protocol 105, and/or dashboards 116 described below. As such, the data devices 104 preferably include a visual display such as a monitor or screen. In some embodiments, the visual display may be incorporated into a web-browser configured to display multimedia content. For instance, a user, such as a patient or a physician, may provide data to data administration tool 106 remotely via an Internet web-browser on a data device 104. Further, the medical or other devices 104e may or may not include a display but may provide data that is incorporated into one or more dashboards or other information summaries provided to a display of another data device 104.

The patient data may include patient-generated data, physician-generated data, provider-generated data, payor-generated data, medical device-generated data, and the like. Patient-generated data comprises patient inputs as to a patient's experiences or the patient's subjective feedback concerning an event, a device, or other similar patient inputs. In some embodiments, patient data device 104a may comprise a browser through which a patient accesses a web-based data collection protocol 105 for gathering or collecting patient-generated data. The web-based data collection protocol 105 may be, e.g., a web-based survey protocol having a log portion, a questionnaire portion, and the like. For instance, a portion of the survey may be a log where the patient can, e.g., rate his or her pain or relative pain level once a day or throughout the day, rate the patient's perceived or subjective recovery level, indicate the patient's activity level, or the like. Another portion of the survey may be configured similar to a questionnaire, presenting the patient with questions or prompts and allowing the patient to select a pre-generated answer or input an answer. That is, the survey may present the patient with answers to choose from, may allow free-form answers, or a combination of both.

Further, the survey may be tailored to the patient's specific procedure or a device the patient is using. As an example, if the patient is using a post-operative infusion pump for regional anesthetic, the survey can present the patient questions or prompts specific to infusion pumps and pain management. The survey may be tailored or customized by a physician or other suitable user of the tracking system 100. For instance, a physician may select to include cardiovascular-related questions for patients receiving cardiovascular care, such as logging the patient's blood pressure and rating chest pain, difficulty breathing, and swelling in certain regions of the patient's body. In some embodiments, the survey protocol has standard or general survey questions, and the physician selects from a library within the tracking system 100 tailored or customized survey questions to include with the standard survey questions when the survey protocol is presented to the patient.

Additionally, the survey protocol may be a dynamic survey protocol that, e.g., presents questions or prompts to the patient based on past responses, past non-responses, or previously collected data. For example, based on an answer by the patient to a previous survey, the survey may ask a follow-up question or omit a question related to the previous answer. That is, the survey may utilize the patient's previous answers or failures to answer to determine current survey questions or prompts. The survey may have other functionalities or configurations as well. In other embodiments, the data collection protocol 105 may be a mobile application, i.e., an app, designed to capture patient inputs, and the app may be used in place of or in addition to the web-based survey to gather patient data. For instance, a patient may download the app onto his or her smartphone before or after a medical procedure to track the patient's recovery, or the patient may download the app as part of tracking the patient's health in general. The app may be configured similarly to the above-described survey, e.g., with a log portion, a questionnaire portion, and the like. In still other embodiments, the data collection protocol 105 may receive patient inputs via email, SMS text messages, or other means of communicating information. The data collection protocol 105 may have other configurations as well.

Moreover, although described above with respect to one patient, it should be appreciated that the tracking system 100 may receive patient-generated data from any number of patients, e.g., through any number and variety of patient data devices 104a. As described in greater detail herein, the tracking system 100 may compile a data set for each individual patient who provides information to the tracking system 100, as well as aggregate each patient data set received by the tracking system 100. The tracking system 100 may analyze the aggregate patient data and, e.g., produce one or more trends for one or more patient populations. For example, from the aggregated patient data, the tracking system 100 may produce a trend for a population of patients utilizing a certain medical device or a population of patients recovering from a certain medical procedure. The tracking system 100 also may compile patient data by physician and/or healthcare organization. For instance, the tracking system 100 may produce one or more trends for all or a subset of patients treated by a certain physician and/or for all or a subset of patients treated at a certain healthcare site or facility.

Physician-generated data comprises physician inputs as to the physician's observations, test results, or similar data relating to the physician's patients. It will be appreciated that “physician” as used herein may refer to any type of caregiver who provides medical care to a patient, whether a medical doctor, a nurse practitioner, a registered nurse, a clinician, or other caregiver. In some embodiments, a physician data device 104b may comprise or be in communication with an electronic medical record (“EMR”) system such that the physician inputs are captured as part of a patient's electronic medical records. In other embodiments, the physician may use a data collection protocol 105, such as one or more mobile applications (i.e., apps), web-based surveys, or the like, to input physician-generated data. Further, although described herein with respect to one physician, it should be understood that any number of physicians, clinicians, or caregivers may be involved in a patient's care and each physician, clinician, or caregiver may generate data specific to the patient that generally is described herein as physician-generated data. Moreover, as described above, the tracking system 100 may receive patient-generated data for any number of patients, and similarly, the tracking system 100 may receive physician-generated data for any number of patients from any number of physicians, clinicians, or caregivers. Provider-generated data may comprise data related to a patient's interaction with a healthcare organization. For example, provider-generated data may include details related to a patient's hospital stay, a patient's interactions with provider personnel upon check-in for an appointment, or the like. In some embodiments, provider inputs through, e.g., a provider data device 104a, may be captured as part of a patient administration system (“PAS”). Provider-generated data may comprise other information as well.

Payor-generated data may comprise data related to a patient's treatment and outcomes or progress. For instance, payor-generated data may include what treatments are available for a given diagnosis, the relative cost of treatment options, the cost of the patient's treatment over time, the patient's outcome to date versus other patients' outcomes using the same treatment for the same amount of time, etc. Other data also may be payor-provided.

Medical device-generated data comprises data from one or more medical devices or instruments 104e used by a patient. Medical device-generated data also may include data from one or more wearable or non-wearable devices, e.g., for detecting the patient's vitals, biofeedback, biomarkers, and/or the patient's activity, such as the patient's heartrate, number of footsteps taken, number of times the patient has stretched a limb (i.e., stretching reps), etc. For example, each medical or other device may have one or more sensors that output data regarding, e.g., the patient's body functions, the device's performance, device usage, or the like. Moreover, although referred to herein together with medical devices or instruments under the term “medical devices,” patient wearable and non-wearable devices may or may not be medically-related, i.e., the devices may or may not be prescribed or monitored by the physician or generally described as a medical device or instrument. Thus, medical device-generated data as used herein generally refers to the inputs, outputs, or data from one or more sensors of medical devices and/or other devices utilized by the patient, such as the wearable and non-wearable devices described above.

As a specific example, a medical device 104e may be an infusion pump that delivers medication to a patient intended to alleviate the patient's pain and that has at least one sensor for sensing the amount of infusion fluid dispensed via the pump over a period of time. As another example, the medical device may include one or more cameras that, e.g., transmit images from the point of view of the camera. Additionally or alternatively, the camera may incorporate GPS tracking and/or motion tracking capabilities, as well as one or more environmental sensors. In one embodiment, a patient's motion may be tracked using one or more accelerometers. Of course, GPS tracking capability, motion tracking capability, and environmental sensors may be incorporated into or onto other medical devices or independently of a medical device or camera. For example, motion tracking capability, such as one or more accelerometers, can be integrated into a wearable device, e.g., a vest, harness, wristband, or the like, and can provide feedback as to a patient's steadiness, limb paresthesia, or other conditions.

As will be readily understood, using data devices 104, the data administration tool 106 may be provided with real-time feedback as to the patient's status and with real-time data regarding the patient's outcomes. For example, the medical device-generated data may be continuously delivered to the data administration tool 106 substantially in real time. Further, using patient data device 104a, the patient can input data at the time of an event or immediately thereafter. Thus, the collection of patient data may occur substantially in real time. It will also be appreciated, as further described below, that the dissemination of the patient data also may occur substantially in real time. Additionally or alternatively, the data administration tool 106 may be integrated with one or more EMR and/or PAS systems, e.g., for sharing of patient data between the data administration tool 106 and the EMR and/or PAS systems. As such, it will be understood that the receipt and/or dissemination may occur substantially in real time, on a specified schedule, or essentially at any time. For instance, the data administration tool 106 may comprise instructions for the receipt and/or dissemination of data according to a schedule, upon satisfaction of one or more conditions, or the like.

The data administration tool 106 is configured to respond to inputs through data devices 104 to provide tracking and management of the patient's outcomes, e.g., the patient's pain management following an outpatient procedure. The data administration tool 106 comprises computer logic utilized to provide the specified functionalities of data administration tool 106. More particularly, as shown in FIG. 1A, the data administration tool 106 comprises a number of processing modules. It will be appreciated that the term “module” refers to computer logic utilized to provide the specified functionality of the module. Thus, a module can be implemented in hardware, firmware, and/or software controlling a general purpose processor. In one embodiment, the modules are program code files stored on the storage device, loaded into memory, and executed by a processor. Alternatively, the modules can be program code files provided from computer program products, e.g., computer executable instructions, which are stored in a tangible computer-readable storage medium such as RAM hard disk or optical or magnetic media. Also, it will be appreciated that embodiments of data administration tool 106 can have different or other modules than the ones described herein, with the described functionalities distributed amongst the modules in a different manner.

Further, the data administration tool 106 may be hosted on a server that is cloud-based, co-located at a provider site, or located at another appropriate site. Additionally, an administrator may have access to the data administration tool 106 to refine the logic or other aspects of the data administration tool 106. It will be appreciated that the administrator may create the logic utilized by data administration tool 106, including the data collection protocol 105 and the various modules of tool 106. The administrator may perform other duties as well, such as managing users, e.g., patients, physicians, etc., of the data administration tool 106.

Data administration tool 106 may respond to the input of patient data by storing the data in one or more databases 108, such as patient information database 108a, communicatively connected to data administration tool 106. The patient information database 108a provides a data repository for the storage and correlation of information gathered from data devices 104. That is, patient information database 108a aggregates, e.g., the patient-generated data, physician-generated data, and other data forming one or more patient data sets as described above. As such, the information stored within the patient information database 108a may be information relating, e.g., to patients' subjective inputs, medical device readings, and test results input by the patients' physician(s). The patient information database 108a may provide information specific or personal to a patient or information regarding a patient population, e.g., a data set devoid of personal identifiers (i.e., de-identified) but identifying a trend among a population of patients. Additionally, as shown in FIGS. 1A and 1B, the data administration tool 106 may be provided access to other databases 108, such as a medical or other device information database 108b, to provide data administration tool 106 with information needed to track and/or manage patient outcomes, develop treatment protocols, or the like. For instance, referring to FIG. 1B, the medical/other device information database 108b may store data from the medical or other devices 104e described above, and database 108b may be linked to database 108a such that database 108b can provide data to data administration tool 106. The data administration tool 106 may be provided access to other databases 108 as well. For example, a clinical database (not shown) may provide information about different therapies that may be used to manage patient recovery. In general, a clinical database provides information that is non-specific or non-personal to a patient, i.e., common information about therapies, devices, and the like without any reference to patient data. In some embodiments, general information databases such as the clinical database may be omitted, and other databases may or may not be provided. It should also be understood that separate databases 108 may be provided for different sets of patient data, e.g., one database 108 may be provided for patient data of one provider, another database 108 may be provided for patient data of another provider, etc., and the data administration tool 106 may access each database 108.

Referring to FIG. 1A, the data administration tool 106 is configured to collect the patient data, e.g., for storage in patient information database 108a, to analyze the patient data, and to disseminate patient data; the data administration tool 106 may have other functionalities as well. More specifically, the data administration tool 106 comprises a collection module 110 for collecting patient data from data devices 104. Collecting the patient data may comprise connecting to one or more data devices 104, e.g., through network 102a as described herein. One or more pieces of patient data may be sent to or received by an analysis module 112 for analysis, which may comprise sorting the data in preparation for analyzing or disseminating the data. In particular, the analysis module 112 includes one or more algorithms to analyze patient behaviors and attitudes, medical device usage, medication usage, and the like to, e.g., perform predictive analytics to trend a patient's outcomes, a patient population's outcomes, or other information regarding a specific patient or a group of patients. For example, analysis module 112, using inputs from a patient data device 104, a physician data device 104b, and/or a medical/other device(s) 104e, may develop a post-operative pain score for a patient, which can be used to develop or modify a treatment protocol for the patient. Further, analysis module 112 may use the patient data to develop other specific therapies, such as, e.g., pain management using a pain pump, enteral feeding using an enteral feeding device, or respiratory health using a respiratory device. As an example, the method and/or system may utilize predictive analytics to provide trending of post-operative pain management based on a patient's use of a pain pump. The analysis module 112 may comprise a rules engine, transformation logic, or the like for performing analysis functions.

The patient data or the results of the analysis of the patient data may be selectively disseminated or distributed via dissemination module 114. At least a portion of the patient data may be available to one or more entities, such as the patient, physician(s), provider(s) or healthcare organization(s), device manufacturer(s), and/or payor(s). In one embodiment, dissemination module 114 is configured to present a customized dashboard 116 to each entity, such that the tracking system 100 comprises a plurality of dashboards 116. For instance, the dissemination module 114 of data administration tool 106 may comprise parameters for each dashboard 116 such that each dashboard 116 displays a visual representation of different patient data or different subsets of patient data. More particularly, as further described herein, access to the patient data may be restricted such that different entities have different levels of access to the patient data. The dashboard parameters may ensure that each dashboard 116 displays only patient data accessible by the entity to which the particular dashboard 116 is provided. Further, the dissemination module 114 may comprise logic or the like for transforming the patient data into a visual representation or summary of the data, such as a graph, chart, or other pictorial representation of the patient data.

In the exemplary embodiment illustrated in FIG. 1A, dissemination module 114 distributes data to a patient dashboard 116a, a physician dashboard 116b, a provider dashboard 116c, a payor dashboard 116d, and a patient population dashboard 116e. As described above, the dashboards 116 may be graphical or other visual representations of patient data, such as charts, graphs, or the like, that may be summaries of the patient data or a portion of the patient data. For example, each patient that interacts with tracking system 100 may have access to a patient dashboard 116a for receiving a summary of the patient's data. The patient dashboard 116a for a specific patient may provide a graphical summary of the patient's survey responses, a trend of the patient's progress toward a health goal, and/or a comparison of the patient's progress to the average progress of a group of patients toward the same health goal. The other dashboards 116 similarly may provide summary information to the targeted entity; for instance, the physician dashboard 116b may provide a physician a summary of an individual patient's data, a summary of the data of a subset of the physician's patients, and/or other data summaries. As further described below, for some entities that have access to one or more dashboards 116, the data administration tool 106 may secure the patient data such that the entity may view only non-identifiable patient data or data summaries, e.g., a trend or summary for one or more patient populations or a trend or summary for an individual patient that does not contain any personal identifiers for the individual patient. Further, one or more dashboards 116 may provide an aggregate benchmark for all patient data within the tracking system 100 that allows an individual physician or a provider to compare patient outcomes against the aggregate benchmark. More specifically, from the patient data for all patients submitting patient data to the tracking system 100 (“the aggregate patient data”), the analysis module 112 may develop aggregate benchmarks within specific filters, e.g., procedure type, demographics, etc. For example, within each filter, the analysis module 112 may average the outcome responses for the aggregate patient data for each survey question of the survey protocol, which is described in greater detail above, and/or for other patient data such as sensor outputs to generate an aggregate benchmark within the filter criteria for each survey question and sensor measure. As a specific example, the analysis module 112 averages the pain score for the aggregate patient data with respect to knee replacement surgery to provide an aggregate benchmark pain score for knee replacement surgery. It will be appreciated that the pain score data may be collected over time, such that the aggregate benchmark pain score for knee replacement surgery may be a trend line over a period of time, e.g., pre-surgery to 90 days post-surgery. Likewise, the analysis module 112 generates a summary, such as a trend line or other appropriate representation of the results of the analysis, for the pain score for knee replacement surgery for a specific patient population, e.g., an individual patient, a subset of a physician's patients, and/or all of that physician's patients. It will be appreciated that the specific patient population is a subset of the aggregate patient population, i.e., the patient data for the specific patient population is a subset of the aggregate patient data.

After the analysis module 112 generates the aggregate benchmark and the specific patient population summary, the dissemination module 114 then may disseminate the aggregate benchmark pain score for knee replacement surgery and the summary of the pain score for knee replacement surgery for the specific population to the physician dashboard 116b such that the physician, e.g., an orthopedic surgeon who performs knee surgeries, can compare an individual patient's pain score, the pain scores of a subset of the physician's patients, or pain scores of all of the physician's patients to the aggregate benchmark. Therefore, using the physician dashboard 116b, the physician can compare patient outcomes against an individual patient group (e.g., the physician's patients) and/or against the larger pool of aggregate patient data to evaluate how the outcomes compare against the aggregate benchmark.

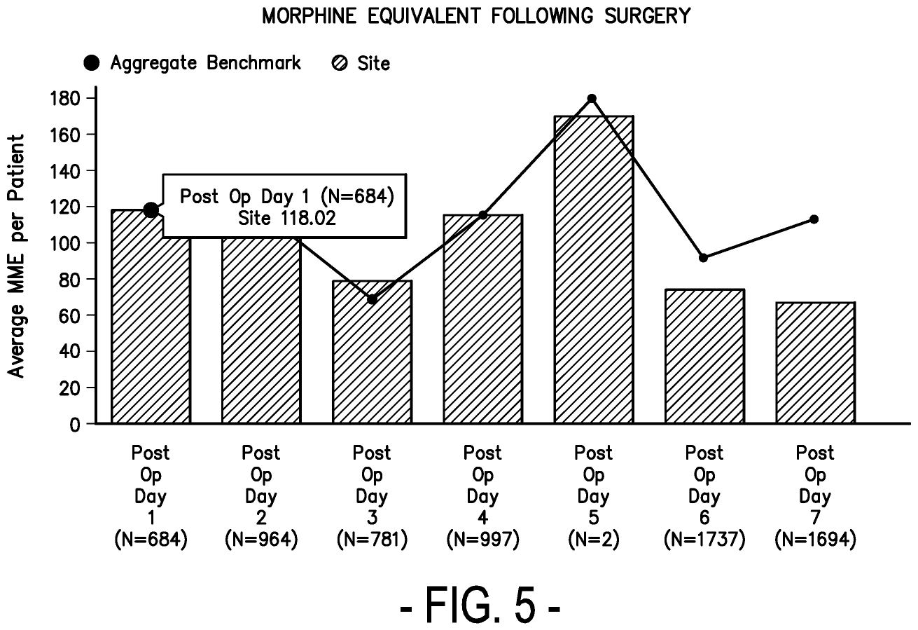

Similarly, a provider such as a hospital administrator can compare outcomes by procedure, physician, etc. at the provider's site against aggregate benchmarks. For example, the analysis module 112 averages a morphine equivalent usage following surgery of patients over time for each physician at a provider's site, as well as an aggregate benchmark of morphine equivalent usage over time by the aggregate patient population (from the aggregate patient data). Morphine equivalent usage may include any number of pain management therapies, such as opioids, infusion pump, etc., with their dosage or use converted to an equivalent morphine dosage. The dissemination module 114 may then disseminate the morphine equivalent usage analysis results to the provider dashboard 116c, which provides a graphical representation of the results, as shown in FIG. 5. Then, using the provider dashboard 116c, the provider can compare morphine equivalent usage of the patients of one physician at the provider's site to the patients of another physician at the provider's site, as well as to the aggregate benchmark morphine equivalent usage. As further depicted in FIG. 5, the provider dashboard 116c also may display the number of patients within the aggregate patient data population provided data in response to a certain survey question or through a certain sensor, e.g., at a certain time. For instance, in the exemplary embodiment shown in FIG. 5, the provider dashboard 116c displays that 684 patients within the aggregate patient data population provided their morphine equivalent usage on the first day following surgery (i.e., Post Op Day 1).

As another example of the comparisons a provider might perform using tracking system 100, the analysis module 112 may generate trend lines by procedure, such that the provider can filter by procedure and view using the provider dashboard 116c a comparison of morphine equivalent usage among procedures (e.g., knee replacement surgery, open heart surgery, etc.) at the provider's site, as well as to the aggregate benchmark morphine equivalent usage for different procedures. Therefore, through tracking system 100, the provider can compare many different measures, such as how physicians within a group (e.g., orthopedic surgeons) at the provider's site relative to one another, how all physicians at the provider's site are performing relative to one another, how one or more physicians at the provider's site are preforming relative to the aggregate patient data (i.e., relative to all physicians whose patient data is within tracking system 100), and/or how the site is performing relative to the aggregate patient data (i.e., relative to all sites whose patient data is within tracking system 100).

Further, the dashboard(s) 116 that present data comparisons or summaries as described above, e.g., physician dashboard 116b and provider dashboard 116c, may be configured to present more than one comparison within the same display. As an example, the analysis module 112 may generate aggregate benchmarks as described above for two or more patient outcomes (such as pain score, sleep duration, morphine equivalent usage, side effects, functional activities, pain management satisfaction, etc.), including up to all of the patient outcomes for which tracking system 100 receives patient data. The dissemination module 114 may disseminate the aggregate benchmarks and other relevant data to, e.g., the physician dashboard 116b, which provides for each patient outcome a graphical representation of a comparison between the patient outcome for a specified patient population and the relevant aggregate benchmark. Multiple graphical representations may appear at the same time within the display of the physician dashboard 116b, i.e., two or more comparisons may appear alongside one another within the display. For instance, for a patient population consisting of the physician's patients, the physician dashboard 116c may provide within the same display a first graphical representation that compares an average of the physician's patients' pain scores over a specified period to a pain score aggregate benchmark; a second graphical representation that compares an average of the physician's patients' sleep duration over a specified period to a sleep duration aggregate benchmark; a third graphical representation that compares an average of the morphine equivalent usage of the physician's patients over a specified period to a morphine equivalent usage aggregate benchmark; and a fourth graphical representation that compares an average of the side effects experienced by the physician's patients over a specified period to a side effects aggregate benchmark. The comparisons or summaries for other patient outcomes may be displayed by selecting to display other data or otherwise signaling to the dashboard 116b to change the display. For example, the comparisons or summaries of the patient outcomes may be on one or more tabs shown on a screen of a graphical user interface, such that the display is changed by selecting a different tab.

Thus, users of the tracking system 100 can compare outcomes among the user's patients, as well as compare outcomes for the user's patients to one or more aggregate benchmarks derived from all patient data within the tracking system 100.

The aggregate benchmarks may be generated and displayed to a user by one or more segments of the tracking system 100 (e.g., analysis module 112 generates aggregate benchmarks, dissemination module 114 disseminates generated aggregate benchmarks, dashboard(s) 116 display aggregate benchmarks). It will be appreciated that any aggregate benchmarks derived from aggregate patient data does not provide patient-identifiable information, such that the aggregate patient data is secured against dissemination of personal information with respect to dissemination of the aggregate benchmarks. Further, using aggregate benchmarks, a caregiver (e.g., a physician, clinician, provider, etc.) can monitor patient outcomes of a variety of patient populations, such as an individual patient or a specified group of patients. By comparing the patient outcomes of a patient population against one or more aggregate benchmarks, the caregiver can assess how the patient population scores against the one or more benchmarks and adjust patient therapies, treatment options, follow-up procedures, etc. as needed. Such comparison may promote on-going quality improvements that improve patient care and, ultimately, improve patient reported outcomes.

Referring to FIG. 1A, the dissemination module 114 also may distribute data to a viewer 118 for viewing individual patient information. That is, the individual patient information viewer 118 may provide raw patient data to an entity authorized to access such data. For example, a physician may be given access to the raw patient data of the physician's patients. The physician may access the physician's patients' data, e.g., by logging in to the individual patient information viewer 118, which grants the physician access to only the physician's patients' data.

As illustrated in FIG. 1A, the data administration tool 106 may include a notification engine 130 for generating one or more notifications based on patient data. For example, within the tracking system 100, clinicians can select to receive notifications that are triggered by a patient's response to one or more survey questions. More particularly, the notification engine 130 may generate two types of notifications, individual or grouped. A clinician determines thresholds for each outcome response for which the clinician wishes to receive a notification, e.g., for each response to a survey question (including standard and custom survey questions) of a survey protocol as described above that could indicate clinician follow-up is needed. Therefore, the thresholds may be customized by each clinician or other appropriate user of the tracking system 100. The notification engine 130 may be part of analysis module 112, dissemination module 114, and/or the dashboard(s) 116 or may be a standalone module within the data administration tool 106.

Individual notifications are triggered if a single outcome response meets or exceeds its threshold, e.g., if pain score or pain management satisfaction or function or sleep or side effects, etc. meets its clinician-selected threshold. Grouped notifications are triggered if a combination of outcome responses meets or exceeds the threshold of each response in the combination, e.g., if responses to two or more specified survey questions meet or exceed the thresholds determined by the clinician. Stated differently, if the clinician selects grouped notifications, a notification will be triggered and sent to the clinician if the patient's responses exceed all the customized thresholds for the outcomes in a group. Thus, grouped notifications may allow the clinician to better customize his or her notification alerts to meaningful events. For instance, a pain score of 7 (single outcome response) may be useful, but a pain score of 7 with a response to pain management satisfaction as very dissatisfied (a combination of outcome responses), may indicate a potential problem for which the clinician should proactively intervene and contact the patient. Another example of grouped notifications may include outcome responses of high opioid use and a large number of side effects, which also could indicate a problem that may warrant clinician intervention. If the clinician has selected to receive a notification if his or her patient gives this combination of outcome responses, the notification engine 130 generates an automatic notification or alert to the clinician. The notification or alert may be delivered via email, SMS message, web portal, or the like. As these examples illustrate, the notification engine 130 enables proactive outreach to address potential care episodes that require medical attention.

In addition to generating notifications based on subjective patient data (i.e., patient-reported outcomes), the notification engine 130 also may generate notifications based on other types of patient data, e.g., data from one or more medical devices 104e. It will be appreciated that the notifications generated from other types of patient data also may be individual notifications or grouped notifications based on clinician-selected thresholds, similar to the individual and grouped notifications described above. As an example, a notification or alert is generated to the clinician if the output from three specified sensors meets or exceeds clinician-selected output thresholds for those sensors. Further, the grouped notifications may extend across data types, e.g., a group of outcome responses may include responses to two survey questions (subjective patient data) and output from a sensor (objective patient data), such as a sensor incorporated into a pain pump.

Moreover, the notifications generated by the notification engine 130 may be assigned amongst a caregiver staff. For example, a clinician may assign the notifications to a nurse, who may follow up with the patient on the clinician's behalf or may further analyze the patient data to formulate an appropriate response. As another example, some outcome responses may be related to the caregiver's facility and best addressed by a manager or administrator of the facility, such that the clinician may assign notifications based on those outcome responses to the manager or administrator. The notifications may be assigned amongst a variety of caregiver staff, e.g., the clinician may receive certain notifications, a nurse may receive other notifications, and a facility manager or administrator may receive still other notifications. Assigning the notifications to particular staff can facilitate effective follow-up.

As described herein, the notifications are configurable in a variety of ways. A user, such as a clinician, can set a threshold for each outcome response or output associated with a patient, or for only those outcome responses or outputs for which the user wishes to receive notifications. The user can select individual or grouped notifications. For grouped notifications, the user can choose the group of outcome responses and/or outputs that trigger a notification. Further, the user can select how to receive the notifications or alerts, e.g., by email, SMS message, or web portal. Additionally, the user can assign the notifications to another caregiver, e.g., a clinician may assign certain notifications to a nurse, certain notifications to a manager of the clinician's facility, etc.

As shown in FIG. 1B, the medical/other device data may be analyzed through a data administration tool 120 that is similar to the data administration tool 106. More particularly, the data administration tool 120 comprises computer logic that is utilized to provide the specified functionalities of data administration tool 120. As shown in FIG. 1B, the data administration tool 120 comprises a collection module 122, an analysis module 124, and a dissemination module 126. Also, it will be appreciated that embodiments of data administration tool 120 can have different or other modules than the ones described herein, with the described functionalities distributed amongst the modules in a different manner. Further, the data administration tool 120 may be hosted on a server that is cloud-based, co-located at a provider site, or located at another appropriate site.

Data administration tool 120 receives data from one or more medical or other devices 104e. The data administration tool 120 may respond to the input of such patient data by storing the data in one or more databases 108, such as medical or other device information database 108b described above, which is communicatively connected to data administration tool 120. The medical/other device information database 108b provides a data repository for the storage and correlation of information gathered from medical or other devices 104e. That is, database 108b aggregates data from one or more medical or non-medical devices 104e, such as medical or other device readings, performance, resets, faults, or the like. Further, as shown in FIGS. 1A and 1B, database 108b may be communicatively connected to patient information database 108a to provide data from devices 104e for use in one or more patient data sets. The medical/other device information database 108b may provide information specific or personal to a patient or information regarding a patient population, e.g., a data set regarding a specific device used by a plurality of patients.

Similar to data administration tool 106, the data administration tool 120 illustrated in FIG. 1B is configured to collect medical or other device data, e.g., for storage in database 108b, to analyze the medical or other device data, and to disseminate such data; the data administration tool 120 may have other functionalities as well. More specifically, the collection module 122 collects patient data from medical or other devices 104e. One or more pieces of such data may be sent to or received by the analysis module 124 for analysis, which may comprise sorting the data in preparation for analyzing or disseminating the data. In particular, the analysis module 124 includes one or more algorithms to analyze, e.g., medical or other device usage, performance, and the like to trend a device's error rate, usefulness in patient treatment, or other information regarding one or more devices. The medical/other device data or the results of the analysis of such data may be selectively disseminated or distributed via dissemination module 126. At least a portion of the medical/other device data may be available to one or more entities, such as the patient, physician(s), provider(s) or healthcare organization(s), medical device manufacturer(s), and/or payor(s). In one embodiment, dissemination module 126 is configured to present a customized dashboard 116f to each device manufacturer. The dashboard 116f may be a graphical or other visual representation of medical/other device data, such as charts, graphs, or the like, that may be summaries of a portion of the device data. The medical/other device data, or summaries of such data, also may be presented through one or more of the dashboards 116 or the patient information viewer 118 illustrated in FIG. 1A.

Preferably, access to the patient data is controlled for each entity. That is, different pieces of the patient data may be accessible to the different entities or different levels of access may be granted to the different entities. As one example, a patient and the patient's physician(s) may be able to access all data related to that patient, but the medical device manufacturer(s) may be able to access only performance data from medical/other device(s) or instrument(s) 104e, which is devoid of any personal data of the patient or any data unrelated to the performance of the device(s). Accordingly, disseminating the patient data may include sorting the data such that the appropriate portion of the data is distributed or made accessible to the appropriate entity.

In some embodiments, patient dashboard 116a is viewable or accessible using patient data device 104a. For example, patient dashboard 116a may be a component of the survey or mobile app that also accepts data inputs by the patient as described above. Alternatively or additionally, the patient may access or view patient dashboard 116a separately from the survey or app, e.g., using the browser of patient data device 104a. Similarly, physician dashboard 116b may be viewable or accessible using physician data device 104b. In other embodiments, patient dashboard 116a and physician dashboard 116b may be standalone components separate from data devices 104a, 104b. Of course, provider dashboard 116c, payor dashboard 116d, and manufacturer dashboard 116f may be viewable or accessible via provider, payor, and manufacturer devices, respectively, that are similar to patient and physician data devices 104a, 104b. In other embodiments, provider dashboard 116c, payor dashboard 116d, and manufacturer dashboard 116f may be standalone components or viewable or accessible from other devices, and such devices may include an interface for the receipt of provider-generated data, payor-generated data, medical/other device-generated data, and manufacturer-generated data. The provider-generated data, payor-generated data, medical/other device-generated data, and manufacturer-generated data may then be viewable or accessible by the patient, the physician, and/or other appropriate entities. Also, the patient population dashboard 116e and individual patient information viewer 118 may be viewable or accessible through one or more data devices 104 or standalone components similar to the dashboards 116a, 116b, 116c, 116d, 116f.

As further depicted in FIG. 1A, data devices 104, data administration tool 106, databases 108, and dashboards 116 are connected and/or multiplexed to network 102a, e.g., via direct network or other suitable links. Similarly, as depicted in FIG. 1B, the medical or other devices 104e, data administration tool 120, database 108b, and manufacturer dashboard 116f are connected and/or multiplexed to network 102b, e.g., via direct network or other suitable links. Preferably, the links in each network 102a, 102b are secure communications channels that include features for preventing tampering with or unauthorized access to the information transmitted using the channels. For example, the communications channels are physically hardened against tampering or are encrypted to prevent unauthorized access to information transmitted thereon. In an exemplary embodiment, the links are secured in a manner that complies with applicable governmental or organizational requirements. As an example, the Health Insurance Portability and Accountability Act (“HIPAA”) regulates access to patient data, and the links may be secured consistent with such regulations to prevent unauthorized access under HIPAA. Accordingly, in an exemplary embodiment, tracking system 100 is HIPAA-compliant.

Similarly, the data administration tools 106, 120 preferably include features for preventing tampering with or unauthorized access to the patient data and, as such, are secure tools. The data administration tools 106, 120 may include such features for securing the patient data in addition to or separately from the features provided to secure the network links as previously described. In some embodiments, a secure data administration tool is one that complies with applicable governmental or organizational regulations. For example, data administration tools 106, 120 may be configured to comply with the requirements of HIPAA such that each data administration tool 106, 120 is HIPAA-compliant. In other embodiments, the data administration tools may include other features or meet other requirements to prohibit tampering with and unauthorized access of the patient data and, thus, be a secure tool. Similar to the network links and data administration tools 106, 120, the patient information database 108a and medical/other device information database 108b may be secured against tampering and unauthorized access and, for example, may conform to HIPAA or other requirements for securing patient data.

It should be appreciated that, in some embodiments, collection module 110, analysis module 112, and/or dissemination module 114 may be separate from data administration tool 106. That is, modules 110, 112, 114 may be standalone components of system 100 in communication with the other components of system 100, e.g., data devices 104, databases 108, and dashboards 116, via network 102a. Similarly, collection module 122, analysis module 124, and/or dissemination module 126 may be separate from data administration tool 120 such that modules 122, 124, 126 may be standalone components of system 100 in communication with the other components of system 100. System 100 may have other configurations as well.

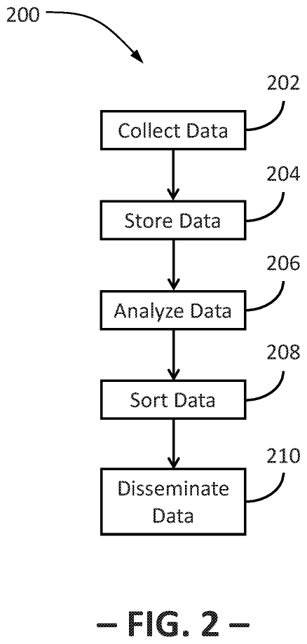

Referring now to FIG. 2, a chart is provided illustrating a method for utilizing tracking system 100, e.g., to track patient outcomes, according to an exemplary embodiment of the present subject matter. As shown, method 200 comprises the steps of collecting patient data and disseminating patient data and also may include the steps of storing, analyzing, and sorting patient data. For example, as described above, patient data may be collected using one or more data devices 104, such as patient data device 104a, physician data device 104b, provider data device 104c, payor data device 104d, and medical/other device 104e. The patient data may then be stored, or it may be analyzed and then stored. Alternatively, the patient data may be sorted then stored, or stored then sorted, and then analyzed. In the illustrated exemplary embodiment, method 200 includes step 202 of collecting patient data; step 204 of storing patient data; step 206 of analyzing patient data; step 208 of sorting patient data; and step 210 of disseminating patient data. In other embodiments, the patient data may be stored, sorted, and/or analyzed in any order and any number of times after it is collected and before it is disseminated. In addition, the patient data may be continually collected and disseminated, with any number and order of storing, sorting, and/or analyzing steps included. Thus, FIG. 2 depicts only an exemplary embodiment of method 200, and the steps of method 200 may be reorganized and repeated as necessary to sufficiently track patient data and thereby track patient outcomes.

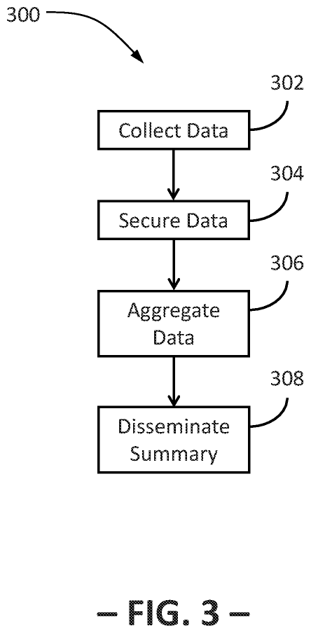

The patient tracking platform may encompass other methods as well. In an exemplary embodiment illustrated in FIG. 3, a method 300 for tracking patient outcomes is provided. The method 300 includes step 302 of collecting patient data of a patient population. The patient population preferably comprises a plurality of patients and, for example, may comprise a plurality of patients who have undergone the same medical procedure or who have a common health goal. The patient data may be inputs received through one or more data devices 104 as described above, and the method for tracking patient outcomes may further comprise inputting the patient data through a plurality of data devices 104. The patient data may be collected by a collection module 110 of a data administration tool 106 as previously described. That is, one or more network connections between the data device(s) 104 and the data administration tool 106 may facilitate the collection of the patient data by the collection module 110.

The method 300 for tracking patient outcomes further comprises aggregating the patient data of the patient population to form an aggregated population data set, as shown at 306 in FIG. 3. The patient data may be aggregated by an analysis module 112 of the data administration tool 106 as described above. Moreover, aggregating the patient data may include compiling, organizing, sorting, etc. the patient data for analysis or for dissemination. The method 300 for tracking patient outcomes also includes disseminating a summary of the aggregated population data set, as shown at 308 in FIG. 3. The summary may be a graphical summary or the like disseminated to one or more dashboards 116 as previously described. In one embodiment, disseminating the summary of the aggregated population data set comprises disseminating a first summary to a first dashboard 116 and disseminating a second summary to a second dashboard 116. Each summary may be tailored to a specific entity, e.g., the first summary may contain some personally identifiable patient information while the second summary does not contain any personally identifiable patient information.

In some embodiments, disseminating the summary of the aggregated population data set comprises disseminating a comparison of two or more therapies. For instance, the patient data may include information regarding the progress of the patients in the patient population with respect to a treatment therapy for attaining a health goal. A database 108 in communication with the data administration tool 106 may contain information regarding another patient population's progress with respect to a different therapy for attaining the same health goal. The summary compares the two patient populations' progress with respect to the two different therapies. Alternatively, the method 300 may comprise, at step 302, collecting patient data of two or more patient populations, where each patient population is subjected to a different therapy. The method 300 may further comprise, at step 306, aggregating the patient data of the patient populations to form aggregated population data sets and disseminating a summary that compares the two or more therapies.

In another embodiment, disseminating the summary of the aggregated population data set comprises disseminating a patient outcome trend of the patient population. For example, the patient data may include information regarding the outcomes of the patients within the patient population. The patient outcome information may be aggregated into a trend such that the trend provides the patients' outcomes over time. As previously described, in some embodiments, the aggregated patient outcome information is an aggregated benchmark, and disseminating the summary of the aggregated population data set comprises disseminating the aggregated benchmark and patient data of a specified patient population such that the patient population is compared to the aggregate benchmark. As described above, an aggregate benchmark may be created for each patient outcome of a patient data set, and each aggregate benchmark may be compared to the corresponding patient outcome for the specified patient population. The method 300 for tracking patient outcomes may further comprise securing the patient data against tampering or unauthorized access, as shown at 304 in FIG. 3. As previously described, the data administration tools 106, 120 and databases 108 that manage the patient data preferably include features for preventing tampering with or unauthorized access to the patient data and, for example, comply with applicable governmental or organizational regulations for securing patient information. In some embodiments, the patient data may be secured in a manner that complies with the requirements of HIPAA. Securing the patient data may include, e.g., assigning different levels of access to different entities. For example, a physician may be provided complete access to patient data of the physician's patients while a medical device manufacturer is provided access to only a portion of the patient data, e.g., a portion of the patient data that does not contain any personally identifiable patient information.

Methods for administering patient data may be provided as well. In an exemplary embodiment, a method for administering patient data includes establishing a survey protocol. The method for administering patient data also may include providing a patient access to the survey protocol and collecting patient data inputs through the survey protocol. In some embodiments, the survey protocol is a web-based survey, but as described above, in other embodiments the survey protocol may be a mobile app downloaded on the patient's smartphone. Further, the survey protocol may be a dynamic survey protocol, which may configure a current survey presented to a patient based on a previous survey presented to the patient, as previously described. In still other embodiments, the survey protocol may include a standard or default set of questions, and a user such as a physician may customize the survey protocol by adding other questions, which may be developed by the physician or selected from a library within the tracking system 100.

The method for administering patient data also may include aggregating patient data inputs collected from a plurality of patients and disseminating a summary of the aggregated patient data inputs. Disseminating the summary of the aggregated patient data inputs may include disseminating a trend of the aggregated patient data. The trend may, e.g., provide a visual representation of any changes in the aggregated patient data over time. Disseminating the summary of the aggregated patient data inputs also may include disseminating a comparison of the aggregated patient data inputs to the patient data inputs of a patient population, e.g., a certain physician's patients.

In some embodiments, the method for administering patient data may further comprise determining a patient procedure for a patient population and configuring, based on the procedure, the survey protocol for collecting patient inputs. The method also may include presenting the patient population with prompts and receiving patient data inputs based on the prompts. The prompts may be questions or statements eliciting one or more responses from the patient as described above with respect to data collection protocol 105. The survey protocol may be presented to each individual patient within the patient population through a patient data device 104a of the individual patient. Further, the patient procedure may be an operation or other medical procedure that each patient of the patient population has undergone. In some embodiments, the patient population may comprise a plurality of patients who have undergone the patient procedure within a certain time frame, e.g., within the past six months or within the past year.

In other embodiments, the method for administering patient data further comprises establishing a first data set to disseminate to a first entity and establishing a second data set to disseminate to a second entity, then disseminating the first data set to the first entity and disseminating the second data set to the second entity. As an example, the first data set may be the patient data of a patient population consisting of a physician's patients, and the second data set may be a portion of the patient data without any personally identifiable information. The first data set may be disseminated to the physician, and the second data set may be disseminated to a medical device manufacturer or a payor organization.

In still other embodiments, the method for administering patient data comprises generating notifications to one or more users based on patient outcomes. As previously described, the notifications may be individual or grouped and are triggered if one or more patient outcomes meet or exceed a threshold defined by a caregiver. For example, a physician may set thresholds for the patient outcomes pain score, morphine equivalent usage, side effects, and sleep duration. If the physician selects to receive individual notifications for each of these patient outcomes, the physician will receive a notification when any one of the patient outcomes meets or exceeds its threshold. If the physician selects to receive grouped notifications, the physician establishes a group of patient outcomes, e.g., morphine equivalent usage and side effects, and the physician will receive a notification if the patient outcomes morphine equivalent usage and side effects both meet or exceed their thresholds. If one patient outcome of a group does not meet or exceed its threshold, the physician does not receive a notification. The method for administering patient data also may comprise assigning the notification(s) to another caregiver.

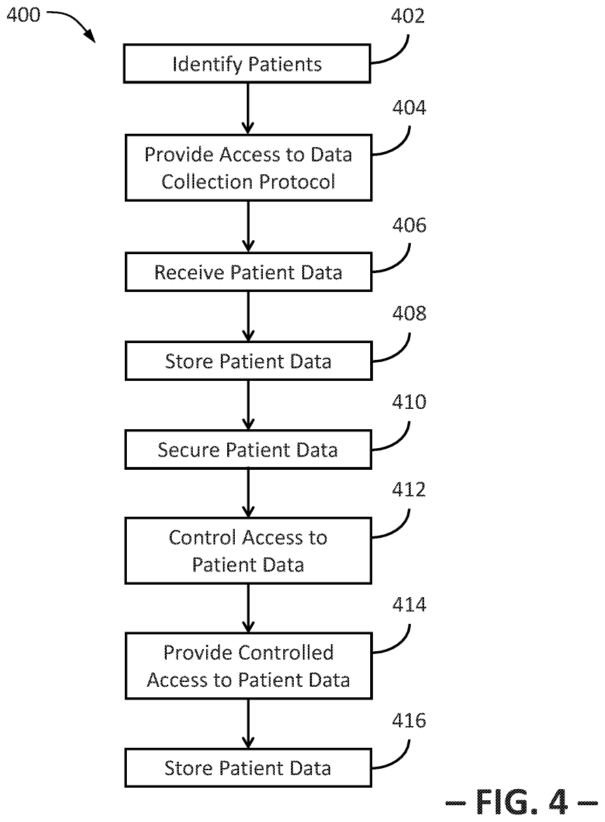

Other methods for administering patient data also may be provided. In one exemplary embodiment illustrated in FIG. 4, a method 400 for administering patient data is provided. The method 400 comprises, at step 402, identifying one or more patients, e.g., an individual patient or a patient population, to receive access to a data collection protocol, such as the data collection protocol 105 previously described. The method 400 further comprises providing the one or more patients access to the data collection protocol and receiving patient data through patient inputs into the data collection protocol, as shown at 404 and 406, respectively, in FIG. 4. Additionally, the method 400 includes steps 408 and 410 of storing and encrypting the patient data (e.g., securing the patient data as previously described), as well as step 412 of controlling access to the patient data, for example, through a data administration tool 106. Storing the patient data may comprise aggregating the patient data to form an aggregated data set. For instance, patient data may be received from a patient population, such that aggregating the patient data includes aggregating the patient data of the patient population to form an aggregated population data set.

The method 400 of administering patient data also includes steps 414 and 416 of providing controlled access to the patient data and disseminating the patient data, e.g., presenting a summary of the aggregated population data set through one or more dashboards 116. In some embodiments, access to the patient data may be controlled by identifying multiple layers of patient data and selectively providing access to one layer, a portion of the layers, or all layers of the patient data, e.g., through a password-protected login system. The layers of patient data may comprise different levels of personal identifiers. For example, a first layer may comprise no personal identifiers, a second layer may comprise some identifying information, a third layer may comprise more personally identifying information than the second layer, and the fourth layer may comprise all personal identifiers of the patient or patients. Different entities, e.g., patients, physicians, providers, payors, and device manufacturers, may be provided different levels of access to the layers of patient data. Further, disseminating the patient data may include disseminating a summary of the patient data by disseminating a comparison of two or more therapies or disseminating a patient outcome trend of the patient population, as described in greater detail above.

The patient tracking platform also may include a method for gathering or collecting patient data. Such method may include, e.g., establishing a procedure a patient has undergone or will undergo; configuring, based on the procedure, a data collection protocol 105, such as an app for patient data device 104a or a web-based survey for display on patient data device 104a; presenting the patient with questions or prompts; and receiving patient input. The platform also may include a method for presenting the questions or prompts to the patient, e.g., comprising selecting appropriate questions or prompts from a database of questions or prompts based on the patient's procedure, the patient's progress, and the intended outcome of the patient. In some embodiments, at least a portion of the questions or prompts may selected by the patient's physician. Further, the questions or prompts may be presented to the patient using patient data device 104a. In some embodiments, the data collection protocol is a dynamic data collection protocol, such that the questions or prompts presented to the patient may change between presentations of the data collection protocol to the patient. That is, the patient may access the data collection protocol on separate occasions, and the data collection protocol may present one or more questions or prompts to the patient that are different from questions or prompts previously presented to the patient.

Further, the patient outcome tracking platform also may include a method for extracting portions or subsets of the patient data for dissemination to different entities. Generally, such method may include determining what data is included in the patient data; establishing a first data set to distribute to a first entity; establishing a second data set to distribute to a second entity; distributing the first data set to the first entity; and distributing the second data set to the second entity. Determining what data is included in the patient data may include determining whether and what personal identifiers are present in the patient data. As such, when the patient data is divided into portions to be disseminated, the personal identifiers can be separated out, or access to the personal identifiers can be restricted, such that the personal identifiers are not disseminated to or accessed by inappropriate entities, e.g., device manufacturers. Of course, it will also be understood that the patient data may be divided into other data sets, e.g., a third data set, a fourth data set, etc., and disseminated to other entities, e.g., a third entity, a fourth entity, etc. Additionally, the same data set may be distributed or disseminated to different entities, e.g., the entire set of an individual patient's patient data may be disseminated to both the patient and the patient's physician, but only a portion of the patient's patient data is disseminated to a payor organization and a different portion or data set is distributed to a device manufacturer.

It will be appreciated that the methods and/or systems described herein provide several benefits or advantages. As one example, the integrated patient data, i.e., the collected and compiled patient-generated, physician-generated, medical device-generated, provider-generated, and payor-generated data generally referred to herein as patient data, can provide insight and trending of post-operative pain management or other health goals for an individual patient or one or more patient populations and thereby be used to influence or develop treatment protocols. More particularly, the patient data, e.g., through trending or the like, can be used to develop or modify treatment protocols for an individual patient or a patient population. As other examples, the methods and/or systems may facilitate the use of the integrated data to allow physicians to compare the effectiveness of various treatment modalities, e.g., in post-operative pain management, or to compare patient outcomes of a patient population to an aggregate benchmark of the patient outcomes. In addition, device manufacturers can use the integrated data, or a portion or subset thereof, to determine the effectiveness of a medical device, areas for improvement in the device (e.g., potential product design improvements), and the device's contribution to a patient's or a patient population's outcome. Further, the integrated data, or a portion or subset thereof, can be used by payor organizations to adjust compensation to physicians based on patient outcomes or outcome trends. Additionally, the methods and/or systems described herein allow clinicians and hospital administrators to track and monitor clinical and post-surgical outcomes, discharge details, readmission, and re-hospitalization, as well as patient satisfaction for one or more patient populations. Moreover, the ability to measure patient reported outcomes via the de-identified data set(s) allows an administrator of a tracking system as described herein to in-service the end users of the system and identify specific procedure recovery trends and actionable insights. Actionable insights can help facilitate discussions of specific results with, e.g., the clinician and hospital administration, to promote on-going quality improvements that improve patient care and patient reported outcomes.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they include structural elements that do not differ from the literal language of the claims or if they include equivalent structural elements with insubstantial differences from the literal language of the claims.

1. A system for monitoring patient outcomes, comprising:
a data device for inputting patient data;
a data administration tool for aggregating the patient data of all patients inputting patient data into the system to form an aggregate patient data set and for generating an aggregate benchmark for a patient outcome from the aggregate patient data set; and
a dashboard for disseminating a comparison of the aggregate benchmark to a summary of the data for the patient outcome of a patient population,
wherein the data for the patient outcome of the patient population is a subset of the aggregate patient data.

2. The system of claim 1, wherein the data administration tool comprises an analysis module that generates the aggregate benchmark.

3. The system of claim 2, wherein the analysis module generates the summary.

4. The system of claim 1, further comprising a notification engine for generating one or more notifications to a caregiver.

5. The system of claim 4, wherein the notification engine is configured to generate a notification when the patient outcome exceeds a threshold.

6. The system of claim 5, wherein the threshold is selected by the caregiver.

7. The system of claim 5, wherein the patient outcome is patient data reported by the patient.

8. The system of claim 1, wherein the data device is a patient data device.

9. The system of claim 8, wherein a patient inputs patient data into a web-based survey accessed through the patient data device.

10. The system of claim 1, wherein the system comprises a plurality of data devices and a plurality of dashboards.

11. A method for tracking patient outcomes, the method comprising:
receiving patient data through patient inputs to a data collection protocol;
aggregating the patient data to form an aggregated patient data set;
generating a first aggregate benchmark for a first patient outcome of the aggregated patient data set; and
disseminating to a dashboard a comparison of a summary of the first patient outcome for a patient population and the first aggregate benchmark.

12. The method of claim 11, further comprising:
generating a second aggregate benchmark for a second patient outcome of the aggregated patient data set; and
disseminating to the dashboard a comparison of a summary of the second patient outcome for the patient population and the second aggregate benchmark,
wherein the dashboard displays the comparison of the summary of the first patient outcome and the first aggregate benchmark alongside the comparison of the summary of the second patient outcome and the second aggregate benchmark.

13. The method of claim 11, further comprising generating a notification based on the patient data.

14. The method of claim 13, wherein the notification is an individual notification that is generated based on a single patient outcome.

15. The method of claim 13, wherein the notification is a group notification that is generated based on a group of patient outcomes.

16. The method of claim 13, wherein the notification is generated when one or more patient outcomes meet or exceed thresholds for the one or more patient outcomes, and wherein the thresholds are selected by a caregiver.

17. A method for administering patient data, the method comprising:
establishing a survey protocol;
providing a patient access to the survey protocol;
collecting patient data inputs through the survey protocol, wherein the patient data inputs represent a plurality of patient outcomes;
aggregating patient data inputs collected from a plurality of patients;
generating an aggregate benchmark for a patient outcome of the aggregated patient data inputs; and
disseminating a comparison of a summary of the patient outcome for a patient population and the aggregate benchmark.

18. The method of claim 17, further comprising:
determining a patient procedure for a patient population;
configuring, based on the patient procedure, the survey protocol for collecting patient inputs;
presenting the patient population with prompts; and
receiving patient data inputs based on the prompts,
wherein configuring the survey protocol comprises including questions or prompts in the survey protocol selected by a caregiver of the patient population.

19. The method of claim 17, further comprising generating a notification based on the patient data, wherein the notification is an individual notification that is generated based on a single patient outcome, and wherein the notification is generated when the single patient outcome meets or exceeds a threshold for the single patient outcome.

20. The method of claim 17, further comprising generating a notification based on the patient data, wherein the notification is a group notification that is generated based on a group of patient outcomes, and wherein the notification is generated when each patient outcome within the group of patient outcomes meets or exceeds a threshold.
